(19) United States
(12) Patent Application Publication
(10) Pub. No.: US 2021/0212852 A1
(43) Pub. Date: Jul. 15, 2021

(54) Devise for the Support of Movement and Lifting Movement of the Human Body

(71) Applicant: Jonas Mast, Baiersbronn (DE)

(72) Inventor: Jonas Mast, Baiersbronn (DE)

(21) Appl. No.: 16/754,180

(22) PCT Filed: Oct. 9, 2018
(86) PCT No.: PCT/EP2018/077492

§ 371 Date: Apr. 7, 2020

(30) Foreign Application Priority Data
Oct. 10, 2017 (DE) 10 2017 123 518.3

(51) Int. Cl. A61F 5/02

(52) U.S. Cl. CPC A61F 5/028; A61F 5/026

(57) ABSTRACT

A device for the support of movement and lifting movement of the human body, which is wearable on the body, with one hip element, with two shoulder straps, with one back element disposed on the hip element and comprising at least one reinforcement element fabricated at least in sections of a shape elastic material, and with two leg straps disposed on the hip element and fabricated at least in sections of an elastomer and disposed on the hip element in such manner that they extend in extension of the back element, wherein each of the leg straps comprises a knee element comprising an upper knee loop and a lower knee loop and a patella element preferably sparing the patella.

The invention relates to a device for the support of movement and lifting movement of the human body, that can be worn on the human body.

While carrying out various activities, many persons, especially industrial employees, are subjected to high physical stresses. Due to inconvenient and awkward postures or continuously repetitive manual activities, hereby the back is especially strongly stressed when lifting and carrying loads. In order to provide support in these cases, devices for the support of movement and lifting movement of the human body are known that are wearable on the body. Such devices are also denoted as exoskeletons.

In this context, active and passive exoskeletons are known. Active exoskeletons are distinguished thereby that they comprise electric motors, hydraulic components or similar systems that, due to their drive, are able to actively support the movements of the joints of the human body. A person consequently experiences direct support during his movements and activities. However, due to the machine elements, active exoskeletons have a high dead weight. In addition, matching the drive of the machine elements to the movement process of the person is difficult since the machine elements must detect at which time which movement process is carried out. Lastly, such active exoskeletons increase the physical bulk of the wearer due to their constructed size and are unwieldy.

Passive exoskeletons do not include drives but rather store potential energy during flexion of the body or of the relevant joint. This energy is released again during extension and supports the relevant movement. Known is, for example, the use of springs. As a rule, the dead weight of passive exoskeletons is less than that of active exoskeletons, and the passive exoskeleton increases the bulk of the wearer less than the active exoskeleton.

The invention addresses the problem of further developing a device, in particular a passive exoskeleton, for the support of movement and lifting movement of the human body such that it comprises several functionalities. It is in particular desirable to provide such a device which, on the one hand, comprises a supporting function of movements, in particular lifting movements, which, however, on the other hand, also comprises a preventive function, in particular by restricting movements that are injurious to health.

According to the invention, the problem is resolved by a device, in particular a passive exoskeleton, for the support of movement and lifting movement of the human body with the characteristics of patent claim 1.

Advantageous implementations and further developments of the invention are specified in the dependent claims.

The device, in particular the passive exoskeleton, according to the invention for the support of movement and lifting movement of the human body wearable on the body, according to the invention comprises one hip element, two shoulder straps, one back element, disposed on the hip element, and at least one reinforcement element, which is, at least in sections, fabricated of a shape elastic material, and two leg straps disposed on the hip element, which are at least in regions fabricated of an elastomer. The leg straps are disposed on the hip element in such manner that they extend in extension of the back element an each comprises a knee element comprising an upper knee loop, a lower knee loop and a patella-sparing patella element.

In the following, by hip element is to be understood an element which, after the device has been fitted to a person, is in contact on the person in the region of the hip. It may be, but does not mandatorily need to be, laid out for securing the device on the person.

In the following, by back element is to be understood an element which, in the state in which the device is fitted to the person, is, at least in sections, in contact on the back of the person. The back element can be unitarily connected to the hip element.

In the following, by shoulder straps are understood those elements which, in the state in which the device is fitted to the person, fix the back element in contact on the back of the person. They can extend from the back across the shoulders to the chest of the person and be guided back to the back underneath the arms. It is, however, also feasible for the back element to rest on the back as well as also on the shoulders and for the shoulder straps not to extend on the shoulder itself but rather substantially on the chest of the person.

Consequently, the device according to the invention is distinguished thereby that, on the one hand, it comprises, at least in sections, elastic components which during flexion of the joints absorb energy and release this energy again during extension in order to support in this manner the relevant movement of the person, which device, on the other hand, however also comprises, at least in sections, shape elastic or, stated differently, quasi rigid or quasi inflexible, components which impede or even prevent movements injurious to health.

For example, when squatting or bending forward the leg straps fabricated of an elastomer, which extend in the extension of the back element, can be stretched in particular through the lengthened path across the buttocks, wherein when rising or straightening up the energy of the elastomers is released again and the rising or straightening-up of the person can thereby be supported.

Yet herein, for example the health-damaging bending from the vertebral column can nearly be prevented due to the reinforcement member if the reinforcement member only permits bending forward with a straight back whereby the intervertebral disks can be relieved. If the reinforcement element restricts torsion of the upper body, painful events under load can be avoided and the person can, for example, be stopped and change the relative position between the momentary position and the load to be lifted or moved.

The knee element according to the invention supports the knee joint, wherein the implementation sparing the patella enables a greater range of motion of the patella and decreases the pressure onto the patella. The leg straps in the disposition according to the invention, extend in particular from the back toward the front across the knee so as to be able to generate a reset force.

The device according to the invention offers overall an advantage in view of support function as well as also prevention. The device according to the invention can herein simultaneously comprise the advantage that, when utilizing very light-weight material, a lower dead weight can be attained. The device can have a very low constructed volume and can thereby be worn on top of clothing as well as also under clothing.

According to an advantageous further development of the invention, the reinforcement element after the device having been fitted to the body, can be oriented substantially parallel to the vertebral column. A reinforcement element disposed such can counteract the flexion of the back beyond a certain angle. It can also counteract the torsion of the upper body beyond a certain angle and/or the lateral tilting movements beyond a certain angle.

The desired effect of the reinforcement can be improved thereby that the back element comprises two or more reinforcement elements.

To keep the dead weight of the device according to the invention as low as possible, the reinforcement element in an advantageous implementation is fabricated of a synthetic material.

According to an especially preferred embodiment of the invention, the shape elastic material has a modulus E of more than 600 N/mm², for example a modulus E of more than 1000 N/mm², in particular a modulus E of approximately 5000 N/mm². Such materials have sufficient rigidity to be able to counteract health-injurious movements. Depending on the modulus E, such materials can, however, simultaneously also have low elasticity so as to be able to offer adequate wear comfort.

An especially preferred embodiment of the invention provides that the reinforcement element is exchangeably disposed on the back element, preferably such that it is slid into a pocket disposed on the back element. On the one hand, this enables the adaptation of the type of reinforcement element to the particular person and, on the other hand, the cleanability of the device can be improved.

To be able to adapt the device in simple manner to the size of the person, the shoulder straps are preferably realized such that they are adjustable in length.

The shoulder straps are each preferably disposed with one end on the back element and with the end either on the back element or the hip element which enables the simple structure of the device.

According to an advantageous embodiment, the hip element is developed as a hip belt whereby a reliable securement of the device on a person can be provided. The hip belt is preferably width-adjustable such that adaptation to the hip circumference of the person that buckles it on can take place.

According to an advantageous implementation of the invention, the elastomer has a modulus E of less than 600 N/mm², for example of less than 150 N/mm², in particular of less than 100 N/mm², for example a modulus E of approximately 30 N/mm². This enables the absorption of sufficient energy during flexing and to offer sufficient support during extension.

Each leg strap is advantageously secured on the hip element such that it is detachable. The leg straps can each be secured on the hip element so as to be individually detachable or also be detachably secured on the hip element across a common securement element. This enables the simpler buckling-on of the device. The leg straps are in particular secured on the hip element in such manner that, when the device is fitted to the person, they extend across the buttocks of the person and are guided to the back of the legs. After the device has been fitted to the person, the leg straps extend, in particular, approximately parallel to the longitudinal axis of the body.

A preferred embodiment of the invention provides for each of the leg straps to be developed such that they are length-adjustable whereby the device can be adapted in simple manner to the size of the person wearing the device.

An advantageous further development of the invention provides for the upper knee loop and/or the lower knee loop to be developed in such manner that they are openable and closable again, preferably by means of a hook-and-loop fastener or other fastening means. This simplifies putting on the knee element since sliding the knee loop from the foot up to the knee area can be avoided.

Each leg strap advantageously comprises a foot element comprising an ankle loop and a sole loop which, in particular, are connected with one another such that they form an 8-shaped element. Such foot element makes it feasible to fix the leg strap reliably on the particular foot. Moreover, such foot element enables good load dissipation across the sole of the foot. The foot element is preferably secured such that it is detachable, which can simplify putting on the device and which enables exchanging the foot elements.

A preferred further development of the invention provides for a heel strap to be disposed between the ankle loop and the sole loop, which heel strap is preferably secured on its regions facing away from one another. Such keel strap can counteract any slipping of the foot element.

The leg straps are preferably developed to be adjustable in their length between the knee element and the foot element in order to be able to adapt the length of the leg straps to the size of a person wearing the device.

The invention will be explained in detail in conjunction with the following Figures. Therein depict:

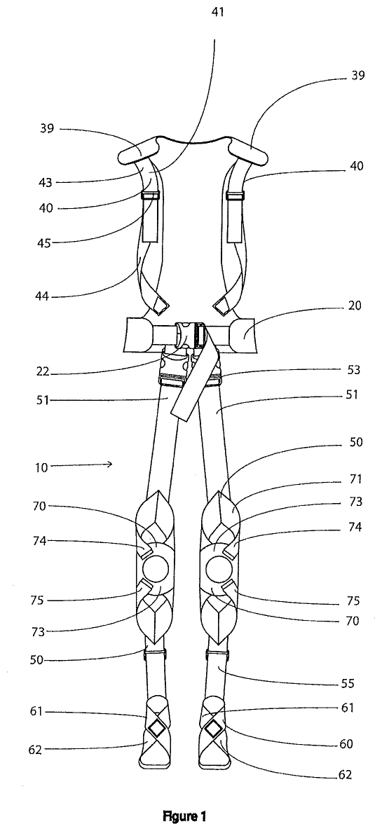

FIG. 1 a view from the front onto an embodiment example of a device according to the invention,

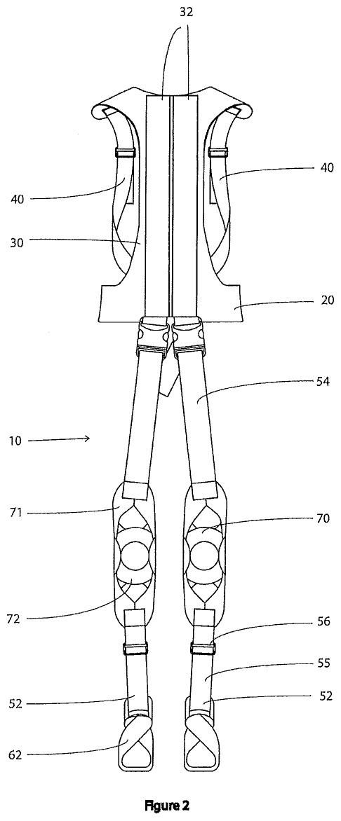

FIG. 2 a view from behind onto the device according to FIG. 1,

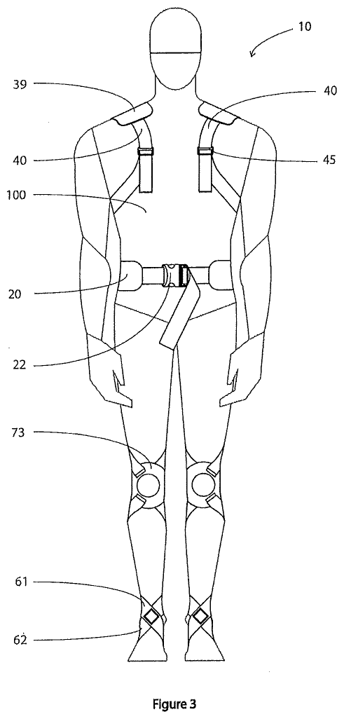

FIG. 3 a view from the front onto the device according to FIG. 1 after it has been fitted to a person,

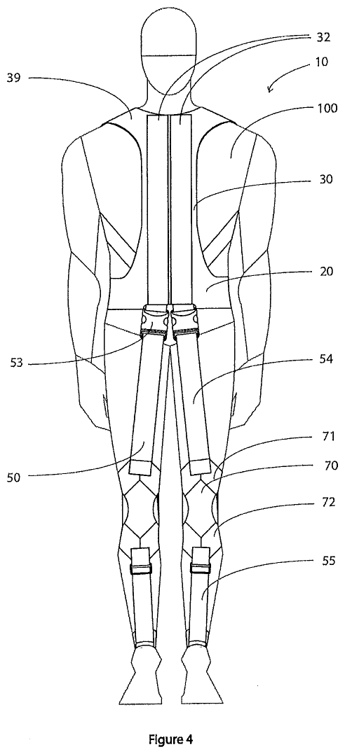

FIG. 4 a view from behind onto the device according to FIG. 1 after it has been fitted to a person,

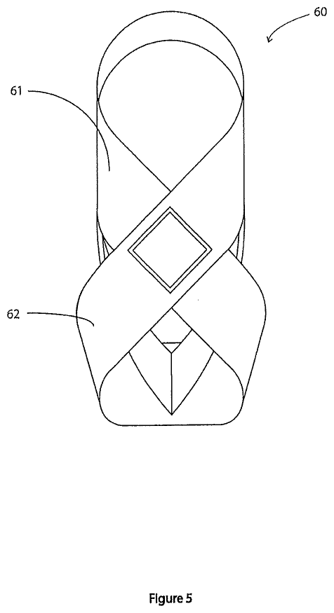

FIG. 5 a view from the front onto an alternative embodiment example of a foot element of a device according to FIG. 1,

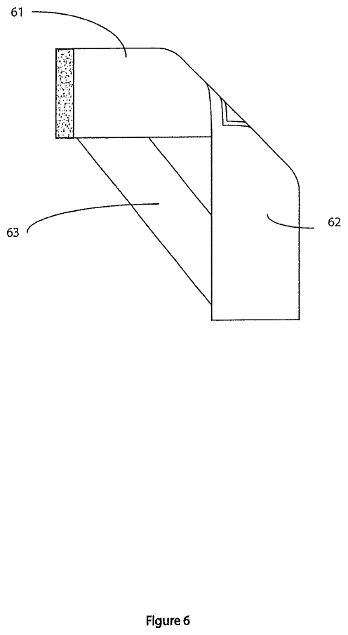

FIG. 6 a side view of the foot element according to FIG. 5, and

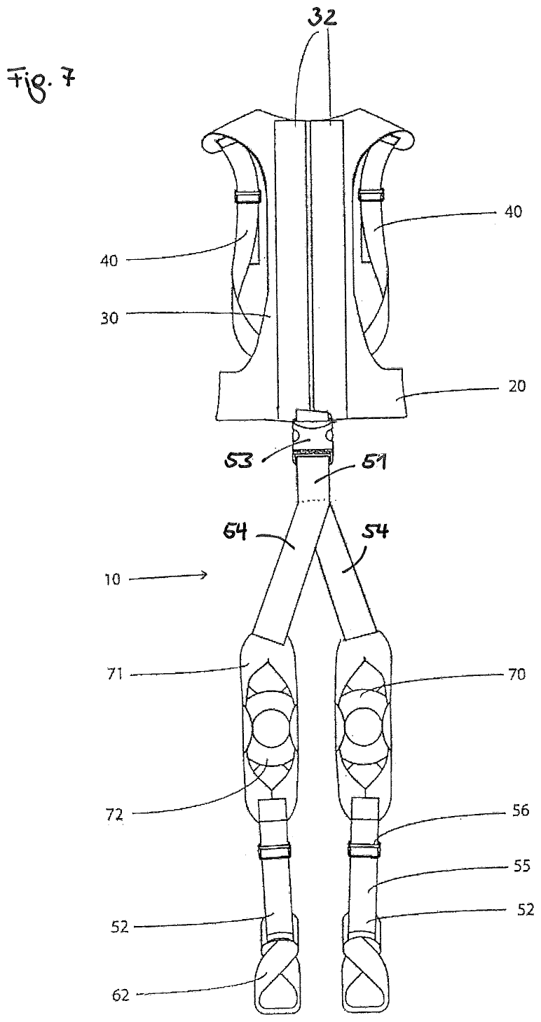

FIG. 7 a view from behind onto a second embodiment example of a device according to the invention.

FIGS. 1 to 4 show various views of an embodiment example of a device 10, in particular of a passive exoskeleton, for the support of movement and lifting movement of the human body. To provide a clearer overview, not all reference numbers are specified in all Figures.

The device 10 comprises a hip element 20 that in the presently discussed embodiment is developed as a hip belt. The hip element 20 can be fixed on a person in the region of the hip, for example thereby that it encompasses the hip. To simplify buckling on the hip element, it can comprise a snap buckle 22 or alternatively also a fastening means in the manner of a belt or a hook-and-loop fastener. Herein, in particular, at least one end of the hip belt is disposed on the snap buckle 22 such that it is length-adjustable in order to be able to adjust the width of the hip element 20.

On the hip element 20 is preferably unitarily disposed a back element 30 which is, in particular of such length that, after it has been fitted to a person 100 (cf. FIGS. 3 and 4), it extends from the region of the hip up into the region of the upper back or neck. The back element 30 can, in particular, also at its end, facing away from the hip element 20, rest with sections 39 on the shoulders of a person 100.

The hip element 20 and the back element 30 can be fabricated of a textile material.

The device 10 comprises two shoulder straps 40, each having a first end 41 and a second end 42. The first end 41 is disposed on the back element 30, for example at the sections 39 of back element 30 resting on the shoulders of the person 100. The second end 42 can be disposed on the hip element 20 or the back element 30 and, in the present embodiment example, is disposed on back element 30 in the proximity of the end facing the hip belt 20. The shoulder straps 40 can be developed as two parts in two sections 43, 44, wherein the one section 43 encompasses the first end 41 and the other section 44 encompasses the second end 42, and the two sections 43, 44 are connected with one another across a buckle 45. The buckle 45 permits in particular the length adjustability of the shoulder straps 40. The shoulder straps 40 can be fabricated of a belt material, for example of a fabric tape.

The device 10 can be fitted to the person 100 thereby that the arms are placed through the loops formed by the back element 30 and the shoulder straps 40 until the back element 30 is in contact on the back, and subsequently the hip belt is closed. With the aid of the buckles 45 the desired length of the shoulder straps 40, and with the aid of the snap buckle 22, the desired width of the hip element 20 can be adjusted until the device 10 has reached a position in which it is fitted to the body and in contact thereon.

The back element 30 comprises at least one reinforcement element 32, for example two reinforcement elements 32, which, after the device 10 has been fitted to the person 100, are oriented, in particular parallel to one another and approximately parallel to the vertebral column. The reinforcement elements 32 can be secured on the outside of back element 30 or be integrated into the back element 30. The reinforcement elements 32 can alternatively also be slid into pockets developed on the back element 30 and thus be developed in particular to be exchangeable.

The reinforcement element 32 is, at least in sections, preferably completely, fabricated of a shape elastic material. The shape elastic material can be, for example, metal or synthetic material, wherein, as a rule, synthetic material is preferred due to its lower weight. The shape elastic material has preferably a modulus E of more than 600 N/mm², especially preferred is a modulus E of more than 1000 N/mm², for example a modulus E of 5000 N/mm².

On the hip element 20 two leg straps 50 are disposed in particular in such manner that they extend in extension of the back element 30, or, stated differently, in particular such that, after the device 10 has been fitted to the person 100, they extend, starting from the hip across the buttocks of person 100 in contact on the legs. The leg straps 50 comprise a first end 51 and a second end 52. The leg straps 50 are preferably detachably disposed with the first end 51 on the hip element 20, for example by means of a snap buckle 53 or alternatively in the manner of a fastener of a belt or by means of a hook-and-loop fastener or other fasteners. The leg strap 50 is herein preferably secured on the snap buckle 53 or the other fastener such that the length adjustability of the leg strap 50 is enabled.

The leg straps 50 are herein disposed either as in the first embodiment example depicted in FIGS. 1 to 4, each detachably individually with the first end 51 on hip element 20 and can be detached or secured each individually. Alternatively, the leg straps 50 can be disposed as in a second embodiment example, depicted in FIG. 7, which only differs from the embodiment example depicted in FIGS. 1 to 4 by the securement of the leg straps 50 on the hip element 20, with their first ends 51 jointly on the hip element 20 and be secured detachably in common, for example by means of a single snap buckle 53 or alternatively in the manner of a fastener of a belt or by means of a hook-and-loop fastener or other fasteners.

At the second end 52 of leg strap 50 a foot element 60 is disposed for securing the second end 52 of the leg strap 50 on a foot of the person 100. The foot element 60 can be secured detachably on the leg strap 50, for example by means of a hook-and-loop fastener. The foot element 60 can comprise an ankle loop 61 and a sole loop 62 which are, in particular, connected with one another such that they form an 8-shaped element. The foot element 60 is slid over the foot of the person 100 such that the ankle loop 61 encompasses the ankle and the sole loop 62 is disposed extending across the back of the foot and the sole of the foot.

An alternative embodiment of the foot element 60 is depicted in FIGS. 5 and 6, wherein the embodiment example depicted here of foot element 60 differs from the embodiment example, depicted in FIGS. 1 to 4, of foot element 60 only by the addition of a heel strap 63. The heel strap 63 is in particular disposed such that, after the person 100 has put on the foot element 60, it extends across the heel up to the sole of the foot. The heel strap 63 preferably connects the ends, facing away from one another, of the ankle loop 61 and the sole loop 62.

The leg strap 50 can comprise a knee element 70. The knee element 70 can comprise an upper knee loop 71, a lower knee loop 72 and a patella element 73, wherein the patella element 73 is preferably developed such that it comprises a void and, after the device 10 has been fitted to the person 100, is developed such that it surrounds the patella. After the person 100 has put on the device 100 [sic: 10] the upper knee loop 71 encompasses the leg of person 100 above the knee, in particular in the lower region of the thigh. After the person 100 has put on the device 10, the lower knee loop 72 encompasses the leg of the person 100 below the knee, in particular in the region of the lower leg.

The patella element 73 encompasses herein the patella and preferably spares it.

The leg strap 50 can be developed in two parts with a first section 54 encompassing the first end 51 and a second section 55 encompassing the second end 52, wherein the knee element 70 is preferably developed between the first section 54 and the second section 55. The second section 55 can be developed to be length-adjustable. For this purpose, the second section 55 can be developed in two parts and the two parts can be connected, for example, by means of a buckle 56.

The leg straps 50 are at least in sections fabricated of an elastomer. The first section 54 and/or the second section 55 can preferably be fabricated of an elastomer. The knee element 70 and/or the foot element [60] can also be fabricated of an elastomer. The elastomer can have in particular a modulus E of less than 600 N.mm², preferably a modulus E of less than 150 N/mm², especially preferred a modulus E of less than 100 N/mm², preferably a modulus E of approximately 30 N/mm².

The leg straps 50 can be put on by the person 100 such that the knee element 70 and the foot element 60 are put on independently of one another. To put on the foot element 60, the foot is guided with the toes pointing forward first through the ankle loop 61 and subsequently through the sole loop 62 until the ankle loop 61 is in contact around the ankle and the sole loop 62 is in contact extending across the back of the foot and the sole of the foot, wherein the connection region between ankle loop 61 and foot loop 62 comes to lie on the back of the foot. In one embodiment the knee element 70 is slipped over the foot and up the leg until the upper knee loop 71 is in contact on the lower region of the thigh and the lower knee loop 72 comes to lie in contact on the upper region of the lower leg. Alternative, it is feasible for the upper knee loop 71 and/or the lower knee loop 72 to be developed such that they are openable and closable again, for example by means of a hook-and-loop fastener or another fastener 74, 75, whereby the fitting is enabled without slipping-up [the knee element 70] starting from the foot. The second section 55 can subsequently be secured on the foot element 60 and the length of section 55 be preferably adapted to the size of the person 100. Lastly, the leg strap 50 is secured on hip element 20, for example by means of the snap buckle 53, wherein the length of the leg strap 50 is preferably adapted to the size of the person 100.

### LIST OF REFERENCE NUMBERS

10 Device
20 Hip element
22 Snap buckle
30 Back element
32 Stiffening element
40 Shoulder strap
41 First end
42 Second end
43 Section
44 Section
45 Buckle
50 Leg strap
51 First end
52 Second end
53 Snap buckle
54 Section
55 Section
60 Foot element
61 Ankle loop
62 Sole loop
63 Heel strap
70 Knee element
71 Upper knee loop
72 Lower knee loop
73 Patella element
74 Fastener
75 Fastener
100 Person

1. A device for support of movement and lifting movement of a human body, which is wearable on the body, comprising:
a hip element, with
two shoulder straps,
a back element disposed on the hip element and comprising a reinforcement element, which at least in sections is a shape elastic material, and
two leg straps, disposed on the hip element, wherein the leg straps are at least in sections of an elastomer and disposed on the hip element such that they extend in extension of the back element,
wherein each leg strap comprises a knee element which comprises an upper knee loop, a lower knee loop and a patella element.

2. The device as in claim 1, wherein the reinforcement element, after the device has been fitted to the body, is substantially oriented parallel to a vertebral column.

3. The device as in claim 1, wherein the back element comprises two or more reinforcement elements.

4. The device as in claim 1, wherein the reinforcement element is a synthetic material.

5. The device as in claim 1, wherein the shape elastic material has a modulus E of more than 600 N/mm².

6. The device as in claim 1, wherein the reinforcement element is disposed on the back element such that it is exchangeable.

7. The device as in claim 1, wherein the shoulder straps are length-adjustable.

8. The device as in claim 1, wherein the shoulder straps are each disposed with one end on the back element and with another end either on the back element or on the hip element.

9. The device as in claim 1, wherein the hip element comprises a width-adjustable hip belt.

10. The device as in claim 1, wherein the elastomer has a modulus E of less than 600 N/mm².

11. The device as in claim 1, wherein each leg strap is detachably secured on the hip element.

12. The device as in claim 1, wherein each leg strap is is length-adjustable.

13. The device as in claim 12, wherein the upper knee loop, the lower knee loop, or both the upper knee loop and the lower knee loop, are openable and closable via a fastener fastener.

14. The device as in claim 1, wherein each leg strap comprises a foot element, secured detachably,
wherein the foot element comprises an ankle loop and a sole loop that are connected with one another in such manner that the ankle loop and sole loop form an 8-shaped element.

15. The device as in claim 14, further comprising a heal strap disposed between the ankle loop and the sole loop, wherein the heal strap is secured at regions of the heal strap facing away from each other.

16. The device as in claim 1, wherein the leg straps are length-adjustable in a region between the knee element and the foot element.
